(19) United States
(12) Patent Application Publication
(10) Pub. No.: US 2025/0049030 A1
(43) Pub. Date: Feb. 13, 2025

(54) METHODS OF SEED AND PLANT TREATMENT TO REDUCE FOODBORNE ILLNESS

(71) Applicant: Ascribe Bioscience Inc., Ithaca, NY (US)

(72) Inventor: Kirk Macolini, Ithaca, NY (US)

(21) Appl. No.: 18/719,942

(22) PCT Filed: Dec. 17, 2022
(86) PCT No.: PCT/US2022/081895

§ 371 Date: Jun. 14, 2024

Related U.S. Application Data
(60) Provisional application No. 63/306,827, filed on Feb. 4, 2022.
(60) Provisional application No. 63/291,334, filed on Dec. 17, 2021.

(51) Int. Cl. A01N 43/16; A01P 1/00

(52) U.S. Cl. CPC A01N 43/16; A01P 1/00

(57) ABSTRACT

This application relates to methods of inhibiting the growth of human pathogenic bacteria in or on a plant by treating the seeds of the plant with an aqueous solution containing one or more ascarosides. The application also provides a method of inhibiting the growth of human pathogenic bacteria in or on a plant by spraying the plant with an aqueous solution comprising one or more ascarosides.

### PRIORITY CLAIM

This application claims priority to U.S. Provisional Patent Application Ser. No. 63/291,334 entitled “Methods of Seed and Plant Treatment to Reduce Foodborne Illness” filed on Dec. 17, 2021 and U.S. Provisional Patent Application Ser. No. 63/306,827 entitled “Methods of Seed and Plant Treatment to Reduce Foodborne Illness” filed on Feb. 4, 2022, both of which are incorporated herein by reference.

### FEDERALLY SPONSORED RESEARCH OR DEVELOPMENT

This invention was made with government support under STTR Grant 1R41AI152915-01 awarded by the National Institute of Health (NIH). The government has certain rights in the invention.

### FIELD OF THE INVENTION

This application generally relates to methods of reducing foodborne illness. More specifically, the invention provides methods of treating seeds or plants to prevent pathogen contamination of edible products derived from such seeds or plants.

### BACKGROUND OF THE INVENTION

Edible sprouts are an increasingly popular food crop, due to year-round production and availability, short growing times, and nutritional benefits that include high levels of vitamins and minerals. However, sprouts are also a high-risk food in terms of frequency of pathogen contamination, and the rise in sprout consumption has been accompanied by a corresponding rise in sprout-related outbreaks of foodborne illnesses. According to the US Centers for Disease Control (CDC) National Outbreak Reporting System, between 1996 and 2017, the U.S. experienced 58 sprout-related foodborne illness outbreaks (31 were multi-state) encompassing at least 1,953 illnesses, 212 hospitalizations, and 5 deaths.

Seeds are a primary source of sprout contamination, with multiple stages of potential pathogen exposure, including seed production, processing, storage, and/or distribution, during which pathogens can enter seeds via cracks, crevices, or intercellular spaces, where they can persist for weeks to years. This renders ineffective common approaches for minimizing pathogen contamination including the application of antimicrobial compounds to irrigation water. Sprout production requires warm and humid conditions that serve to further amplify the growth of any contaminating bacterial pathogens. Thus, even low bacterial levels in the seed can result in high microbial loads by the end of the sprout production process. In most cases, there are no visible symptoms that a sprout is contaminated with pathogens. Additionally, biofilm formation by both nonpathogenic and pathogenic bacteria can occur on the exterior or interior of the sprout and provide a protected environment where pathogenic bacteria can colonize and thrive. Thus, pathogenic bacterial contamination of sprouts is not easily addressed. Furthermore, antimicrobial treatments can adversely affect germination rates and yield of sprouts and are therefore not attractive to growers and the sprout industry. These problems can also affect plants that are grown from the contaminated seeds.

The FDA has made considerable investments to mitigate food safety hazards related to edible sprouts. In 2019, the FDA issued the draft guidance, “Reducing Microbial Food Safety Hazards in the Production of Seed for Sprouting,” with specific recommendations to help reduce food safety hazards. It states that seeds for sprouting are “food,” and to the extent possible, must be handled as such. Unfortunately, seed growers do not always know whether the seed they are producing will be used for sprout production. Therefore, the seed may be exposed to manure fertilizers or other contaminating sources (rodents, birds) during production that, while having little to no impact on mature field crops grown from this seed, pose a serious threat to hygienic sprout production.

According to the FDA, no single treatment has been shown to eliminate human pathogenic bacteria on seeds or sprouts, and those treatments that can significantly reduce sprout contamination negatively affect germination and vigor. Therefore, every precautionary measure should be taken to prevent high levels of bacterial contamination, including all steps involved in seed to sprout production. The FDA recommends that seeds used for edible sprouting should be subjected to one or more treatments to reduce the levels of Salmonella and E. coli O157 by at least 5 logs. Achieving this level of reduction is a daunting task. Extensive washing and chlorine treatments have proven ineffective in sanitizing sprout seeds because microbes can reside in protected niches on the seed surface or under the seed coat. While researchers have attempted various approaches to improve the microbial safety of sprouts including treatments with electrolyzed water, organic acids, calcium hypochlorite, or sodium hypochlorite (bleach), none of these treatments are sufficiently effective in eliminating human pathogenic bacteria from sprouts. Additionally, both calcium and sodium hypochlorite are toxic by oral ingestion and dermal contact and their solutions can release toxic gases, potentially putting workers at risk. Worse still, these chemicals are strong oxidizing and halogenating agents and may degrade the nutritive value of sprouts or contaminate them with residual chlorinated organic molecules formed during treatment. It would be beneficial to provide safer and more effective methods to ensure the safety of sprouts as a food source.

### SUMMARY OF THE INVENTION

Compositions and methods for inhibiting pathogen growth in seeds, plants, or plant parts are provided. Compositions comprise at least one ascaroside. In some embodiments, the composition comprises more than one ascaroside as well as additional components to enhance pathogen inhibition. In one embodiment, the additional component comprises salicylic acid. Methods of the invention include soaking, spraying, coating, contacting seeds and/or sprouts of a plant to prevent or inhibit pathogen growth inside the growing plant.

In an embodiment, the one or more ascarosides conform to Formula A.:

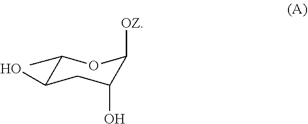

where Z is:
(i) —CH(CH₃)—R¹, where R¹ is an optionally substituted C₁₋₄₀ aliphatic group;
(ii) —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide;
(iii) —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide;
(iv) —CH(CH₃)—(CH₂)ₙ—CH(OH)—CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide;
(v) —(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide;
(vi) —(CH₂)ₙ—CH═CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide; or
(vii) —(CH₂)ₙ—CH(OH)—CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

In a particular embodiment, for ascarosides of formula A, Z is —CH(CH₃)—(CH₂)ₙ CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide. A specific example of this type of ascaroside is ascr #18, shown below.

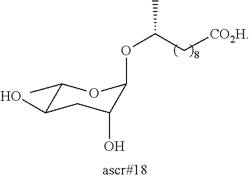

In a particular embodiment, the one or more ascarosides have formula (A) wherein Z is —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide. A specific example of this type of ascaroside is ascr #7, shown below.

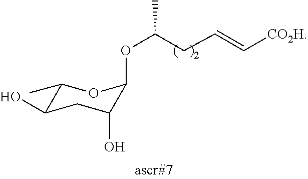

In an embodiment, the step of treating seeds of the plant with an aqueous solution comprising one or more ascarosides comprises soaking the seeds in the aqueous solution. In one embodiment, the seeds are soaked in the aqueous solution for a time of about 1 min to about 6 hours. The concentration of the one or more ascarosides in the aqueous solution may be about 0.001 mM to about 10.0 mM. In an embodiment, the seeds are soaked in the aqueous solution for a period of time sufficient to bring the seeds out of quiescence.

In a further aspect a method is provided of enhancing the growth of a plant. In certain embodiments, the method comprises treating seeds of the plant with an aqueous solution comprising one or more ascarosides, wherein plants grown from the treated seeds have accelerated growth compared to plants grown from untreated seeds. The one or more ascarosides can have the structure as set forth in the other embodiments provided hereinabove.

In an additional aspect a method is provided of producing edible sprouts. In certain embodiments, the method comprises: providing seeds from a plant that can be used to produce edible sprouts; treating the seeds by soaking them in an aqueous solution comprising one or more ascarosides; placing the seeds in a container suitable for sprouts to form from the treated seeds; and harvesting the sprouts after the sprouts reach a desired size. Again, the one or more ascarosides can have the structure as set forth in other embodiments described herein above.

The invention includes, without limitation, the following embodiments:

Embodiment 1: A method of inhibiting the growth of human enteric pathogens in a plant, comprising treating seeds of the plant with a composition comprising one or more ascarosides.

Embodiment 2: The method of embodiment 1, wherein the one or more ascarosides have the structure (I)

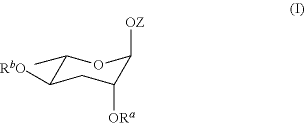

where: Z is an optionally substituted C₃₋₄₀ aliphatic group, and each of R^{a} and R^{b} is independently —H, or an optionally substituted moiety selected from the group consisting of: C₁₋₂₀ aliphatic, C₁₋₂₀ acyl, C₁₋₂₀ heteroaliphatic, aryl, heteroaryl, a hydroxyl protecting group, a phosphorous-linked functional group, a sulfur-linked functional group, a silicon-linked functional group, a C₂₋₂₀ carbonate (e.g., a moiety —C(O)OR^{c}), a C₂₋₂₀ carbamate (e.g., a moiety —C(O)N(R^{c})₂), a C₂₋₂₀ thioester (e.g., a moiety —C(S)R^{c}), a C₂₋₂₀ thiocarbonate (e.g., a moiety —C(S)OR^{c}), a C₂₋₂₀ dithiocarbonate (e.g., a moiety —C(S)SR^{c}), a C₁₋₂₀ thiocarbamate (e.g., a moiety —C(S)N(R^{c})₂), a sugar moiety, a peptide, a polymer chain, or a linkage via a bond or a carbon-containing linker moiety to another ascaroside molecule. Where R^{c} is independently at each occurrence selected from —H, optionally substituted C₁₋₁₂ aliphatic, optionally substituted C₁₋₁₂ heteroaliphatic, optionally substituted aryl, optionally substituted heteroaryl, a polymer chain, or a linkage via a bond or a carbon-containing linker moiety to another ascaroside molecule, and where R^{a} and R^{b} may be taken together to form an optionally substituted ring, optionally containing one or more heteroatoms, and optionally containing one or more sites of unsaturation.

Embodiment 3: The method of embodiment 2, wherein Z is selected from the group consisting of: —CH(CH₃)—R¹, where R¹ is an optionally substituted C₁₋₄₀ aliphatic group; —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide; —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide; —CH(CH₃)—(CH₂)ₙ—CH(OH)—CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide; —CH(CH₃)—(CH₂)ₙ—C(O)—CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide; —(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide; —(CH₂)ₙ—CH═CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide; —(CH₂)ₙ—CH(OH)—CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide; and —(CH₂)ₙ—C(O)—CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

Embodiment 4: The method of embodiment 2 or 3, wherein R^{a} and R^{b} are each —H.

Embodiment 5: The method of any of embodiments 2-4, wherein Z is —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

Embodiment 6: The method of embodiment 1, wherein at least one of the one or more ascarosides is ascr #18.

Embodiment 7: The method of any of embodiments 2-4, wherein Z is —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

Embodiment 8: The method of embodiment 1, wherein at least one of the one or more ascarosides is ascr #7.

Embodiment 9: The method of any of embodiments 1-8, wherein treating seeds of the plant comprises soaking the seeds in the aqueous solution comprising one or more ascarosides.

Embodiment 10: The method of embodiment 9, wherein the seeds are soaked in the aqueous solution for a time of about 1 min to about 6 hours.

Embodiment 11: The method of claim 9, wherein the concentration of the one or more ascarosides in the aqueous solution is between about 0.001 mM and about 1.0 mM.

Embodiment 12: The method of any of claims 1-11, wherein the seeds of the plant are seeds of a plant that can be used to produce edible sprouts.

Embodiment 13: The method of embodiment 12, wherein the seeds of the plant are seeds that can produce edible legume family sprouts.

Embodiment 14: The method of embodiment 12, wherein the seeds of the plant are seeds that can produce edible cereal family sprouts.

Embodiment 15: The method of embodiment 12, wherein the seeds of the plant are seeds that can produce edible oilseed family sprouts.

Embodiment 16: The method of embodiment 12, wherein the seeds of the plant are seeds that can produce edible cabbage family sprouts.

Embodiment 17: The method of embodiment 12, wherein the seeds of the plant are seeds that can produce edible parsley family sprouts.

Embodiment 18: The method of embodiment 12, wherein the seeds of the plant are seeds that can produce edible onion family sprouts.

Embodiment 19: The method of embodiment 12, wherein the seeds of the plant are seeds that can produce edible vegetable or herb sprouts.

Embodiment 20: The method of embodiment 12, wherein the seeds of the plant are seeds that produce crop plants.

Embodiment 21: The method of any of embodiments 9-20, wherein the seeds are soaked in the aqueous solution for a period of time sufficient for the seeds to gain about 2% to about 20% of their dry weight.

Embodiment 22: The method of any of embodiments 9-21, wherein the seeds are soaked in the aqueous solution for a period of time sufficient to bring the seeds out of quiescence.

Embodiment 23: A method of inhibiting the growth of human enteric pathogens in or on a plant comprising spraying the plant with a composition comprising one or more ascarosides.

Embodiment 24: The method of embodiment 23, wherein the one or more ascarosides have the structure (I)

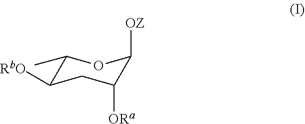

where: Z is an optionally substituted C₃₋₄₀ aliphatic group, and each of R^{a} and R^{b} is independently —H, or an optionally substituted moiety selected from the group consisting of: C₁₋₂₀ aliphatic, C₁₋₂₀ acyl, C₁₋₂₀ heteroaliphatic, aryl, heteroaryl, a hydroxyl protecting group, a phosphorous-linked functional group, a sulfur-linked functional group, a silicon-linked functional group, a C₂₋₂₀ carbonate (e.g., a moiety —C(O)OR^{c}), a C₂₋₂₀ carbamate (e.g., a moiety —C(O)N(R^{c})₂), a C₂₋₂₀ thioester (e.g., a moiety —C(S)R^{c}), a C₂₋₂₀ thiocarbonate (e.g., a moiety —C(S)OR^{c}), a C₂₋₂₀ dithiocarbonate (e.g., a moiety —C(S)SR^{c}), a C₁₋₂₀ thiocarbamate (e.g., a moiety —C(S)N(R^{c})₂), a sugar moiety, a peptide, a polymer chain, or a linkage via a bond or a carbon-containing linker moiety to another ascaroside molecule. Where R^{c} is independently at each occurrence selected from —H, optionally substituted C₁₋₁₂ aliphatic, optionally substituted C₁₋₁₂ heteroaliphatic, optionally substituted aryl, optionally substituted heteroaryl, a polymer chain, or a linkage via a bond or a carbon-containing linker moiety to another ascaroside molecule, and where R^{a} and R^{b} may be taken together to form an optionally substituted ring, optionally containing one or more heteroatoms, and optionally containing one or more sites of unsaturation.

Embodiment 25: The method of embodiment 24, wherein R^{a} and R^{b} are both —H.

Embodiment 26: The method of embodiment 24 or 25, wherein Z is selected from the group consisting of —CH(CH₃)—R¹, where R¹ is an optionally substituted C₁₋₄₀ aliphatic group; —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide; —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide; —CH(CH₃)—(CH₂)ₙ—CH(OH)—CH—CO₂R³², where n is an integer from 1 to 40, and R² is —H, a metal cation, an optionally substituted C₁₋₂₀ aliphatic group, an optionally substituted aromatic group, a glycoside, an amino acid, a peptide, or a nucleotide; —CH(CH₃)—(CH₂)ₙ—C(O)—CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide; —(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide; —(CH₂)ₙ—CH═CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide; —(CH₂)ₙ—CH(OH)—CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide; and —(CH₂)ₙ—C(O)—CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

Embodiment 27: The method of embodiment 24 or 25, wherein Z is —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

Embodiment 28: The method of embodiment 23, wherein at least one of the one or more ascarosides is ascr #18.

Embodiment 29: The method of embodiment 24 or 25, wherein Z is —CH(CH₃)—(CH₂)ₙ—CH═CH—CO₂R³, where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

Embodiment 30: The method of embodiment 23, wherein at least one of the one or more ascarosides is ascr #7.

Embodiment 31: The method of any of embodiments 23-30, wherein a concentration of the one or more ascarosides in the composition is about 1 ppb to about 50 ppm.

Embodiment 32: The method of any of embodiments 23-31, wherein the plant is an edible sprout plant.

Embodiment 33: The method of embodiment 32, wherein the edible sprout plant is an edible legume family sprout plant.

Embodiment 34: The method of embodiment 32, wherein the edible sprout plant is an edible cereal family sprout plant.

Embodiment 35: The method of embodiment 32, wherein the edible sprout plant is an edible oilseed family sprout plant.

Embodiment 36: The method of embodiment 32, wherein the edible sprout plant is an edible cabbage family sprout plant.

Embodiment 37: The method of embodiment 32, wherein the edible sprout plant is an edible parsley family sprout plant.

Embodiment 38: The method of embodiment 32, wherein the edible sprout plant is an edible onion family sprout plant.

Embodiment 39: The method of embodiment 32, wherein the edible sprout plant is an edible vegetable or herb sprout plant.

Embodiment 40: The method of any of embodiments 23-31, wherein the plant is a crop plant.

Embodiment 41: A method of producing edible sprouts comprising: treating seeds from a plant that can be used to produce edible sprouts by soaking the seeds in an aqueous solution comprising one or more ascarosides; placing the treated seeds in a container suitable for allowing sprouts to form from the treated seeds; and harvesting the sprouts after the sprouts reach a suitable size

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise. Other aspects and advantages of the present disclosure will become apparent from the following.

### Definitions

In order for the present disclosure to be more readily understood, certain terms are first defined below. Additional definitions for the following terms and other terms are set forth throughout the specification.

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. As used in this application, the term “comprise” and variations of the term, such as “comprising” and “comprises,” are not intended to exclude other additives, components, integers or steps.

About, Approximately: As used herein, the terms “about” and “approximately” are used as equivalents. Unless otherwise stated, the terms “about” and “approximately” may be understood to permit standard variation as would be understood by those of ordinary skill in the art. Where ranges are provided herein, the endpoints are included. Any numerals used in this application with or without about/approximately are meant to cover any normal fluctuations appreciated by one of ordinary skill in the relevant art. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

Certain compounds provided herein can comprise one or more asymmetric centers, and thus can exist in various stereoisomeric forms, e.g., enantiomers and/or diastereomers. Thus, inventive compounds and compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of enantiomers or diastereomers are provided.

Furthermore, certain compounds as described herein may have one or more double bonds that can exist as either a Z or E isomer, unless otherwise indicated. The compounds can be provided as individual isomers substantially free of other isomers and alternatively, as mixtures of various isomers, e.g., racemic mixtures of enantiomers.

As used herein, the term “isomers” includes any and all geometric isomers and stereoisomers. For example, “isomers” include cis- and trans-isomers, E- and Z-isomers, R- and S-enantiomers, diastereomers, (D)-isomers, (L)-isomers, racemic mixtures thereof, and other mixtures thereof, as falling within the scope of the invention. For instance, a compound may, in some embodiments, be provided substantially free of one or more corresponding stereoisomers, and may also be referred to as “stereochemically enriched.”

Where a particular enantiomer is preferred, it may, in some embodiments be provided substantially free of the opposite enantiomer, and may also be referred to as “optically enriched.” “Optically enriched,” as used herein, means that the compound is made up of a significantly greater proportion of one enantiomer. In certain embodiments the compound is made up of at least about 90% by weight of an enantiomer. In some embodiments the compound is made up of at least about 95%, 97%, 98%, 99%, 99.5%, 99.7%, 99.8%, or 99.9% by weight of an enantiomer. In some embodiments the enantiomeric excess of provided compounds is at least about 90%, 95%, 97%, 98%, 99%, 99.5%, 99.7%, 99.8%, or 99.9%. In some embodiments, enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, IN 1972).

The terms “halo” and “halogen” as used herein refer to an atom selected from fluorine (fluoro, —F), chlorine (chloro, —Cl), bromine (bromo, —Br), and iodine (iodo, —I).

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-30 carbon atoms. In certain embodiments, aliphatic groups contain 1-12 carbon atoms. In certain embodiments, aliphatic groups contain 1-8 carbon atoms. In certain embodiments, aliphatic groups contain 1-6 carbon atoms. In some embodiments, aliphatic groups contain 1-5 carbon atoms, in some embodiments, aliphatic groups contain 1-4 carbon atoms, in yet other embodiments aliphatic groups contain 1-3 carbon atoms, and in yet other embodiments aliphatic groups contain 1-2 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “heteroaliphatic” or “heteroaliphatic group”, as used herein, denotes an aliphatic group where one or more carbon or hydrogen atoms are replaced by a heteroatom (e.g., oxygen, nitrogen, sulfur, phosphorous, boron, etc.).

The term “unsaturated”, as used herein, means that a moiety has one or more double or triple bonds.

The term “alkyl,” as used herein, refers to saturated, straight- or branched-chain hydrocarbon radicals derived from an aliphatic moiety containing between one and six carbon atoms by removal of a single hydrogen atom. Unless otherwise specified, alkyl groups contain 1-12 carbon atoms. In certain embodiments, alkyl groups contain 1-8 carbon atoms. In certain embodiments, alkyl groups contain 1-6 carbon atoms. In some embodiments, alkyl groups contain 1-5 carbon atoms, in some embodiments, alkyl groups contain 1-4 carbon atoms, in yet other embodiments alkyl groups contain 1-3 carbon atoms, and in yet other embodiments alkyl groups contain 1-2 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

The term “alkenyl,” as used herein, denotes a monovalent group derived from a straight- or branched-chain aliphatic moiety having at least one carbon-carbon double bond by the removal of a single hydrogen atom. Unless otherwise specified, alkenyl groups contain 2-12 carbon atoms. In certain embodiments, alkenyl groups contain 2-8 carbon atoms. In certain embodiments, alkenyl groups contain 2-6 carbon atoms. In some embodiments, alkenyl groups contain 2-5 carbon atoms, in some embodiments, alkenyl groups contain 2-4 carbon atoms, in yet other embodiments alkenyl groups contain 2-3 carbon atoms, and in yet other embodiments alkenyl groups contain 2 carbon atoms. Alkenyl groups include, for example, ethenyl, propenyl, butenyl, 1-methyl-2-buten-1-yl, and the like.

The term “aryl” used alone or as part of a larger moiety as in “aralkyl”, “aralkoxy”, or “aryloxyalkyl”, refers to monocyclic and polycyclic ring systems having a total of five to 20 ring members, wherein at least one ring in the system is aromatic and wherein each ring in the system contains three to twelve ring members. The term “aryl” may be used interchangeably with the term “aryl ring”. In certain embodiments of the present invention, “aryl” refers to an aromatic ring system which includes, but is not limited to, phenyl, biphenyl, naphthyl, anthracyl and the like, which may bear one or more substituents. Also included within the scope of the term “aryl”, as it is used herein, is a group in which an aromatic ring is fused to one or more additional rings, such as benzofuranyl, indanyl, phthalimidyl, naphthimidyl, phenantriidinyl, or tetrahydronaphthyl, and the like.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄R^{o}; —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)N(R^{o})₂; —(CH₂)₀₋₄C(O)SR^{o}; —(CH₂)₀₋₄C(O)OSiR^{o}₃; —(CH₂)₀₋₄C(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₈ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or polycyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₄C(O)N(R^{o})₂; —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═0 and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “substantially” refers to the qualitative condition of exhibiting total or near-total extent or degree of a characteristic or property of interest.

The term “sprout” as used herein is the plant material that is produced from seeds after germination of the seed, but prior to formation of leaves on the plant material. “Edible sprouts” are sprouts that are considered non-toxic to humans.

The convention of naming ascarosides by a several-letter prefix followed by a pound sign (#) and a number is sometimes used (for example ascr #18). This convention is used in the scientific literature and the skilled artisan will understand that each such name is associated with a specific chemical structure of known composition and will readily apprehend the structure of the molecule referred to using this naming convention. Unless otherwise indicated, all compound identifiers of this format used herein conform to the definitions described in the C. elegans Small Molecule Identifier Database (SMID-DB) maintained at http://www.smid-db.org.

### BRIEF DESCRIPTION OF THE DRAWINGS

In the drawings, the features are not necessarily represented to scale, emphasis instead generally being placed upon illustrating the principles of the disclosed compositions and methods and are not intended as limiting. For purposes of clarity, not every component may be labeled in the drawing. In the following description, various embodiments are described with reference to the following drawings, in which:

FIG. 1 depicts a bar graph comparing the effect of treatment of alfalfa seeds with an ascaroside solution on the Salmonella counts on sprouts produced from the treated alfalfa seeds.

FIG. 2 depicts a bar graph comparing the effect of treatment of seeds with an ascaroside solution on the upregulation of plant defense genes, compared to a control sample.

FIG. 3A depicts a bar graph comparing the effect of spraying rice seeds with an ascaroside solution on the emergence of rice plants.

FIG. 3B depicts a bar graph comparing the effect of spraying rice seeds with an ascaroside solution on the height of rice plants.

FIG. 4A shows the effect of ascr #18 treatment of tomato plant (cv. Sweet hybrid 100) seeds on lesion size caused by an oomycete pathogen.

FIG. 4B shows the effect ascr #18 treatment of tomato plant (cv. M82) seeds on lesion size caused by an oomycete pathogen.

### DETAILED DESCRIPTION OF THE INVENTION

Compositions and methods for inhibiting pathogen growth in seeds, plants, or plant parts are provided. Compositions comprise at least one ascaroside. Additional components include agriculturally acceptable carriers and components to enhance pathogen inhibition, or components to enhance sprout formation or growth. Methods of the invention include soaking, spraying, coating, contacting seeds and/or sprouts of a plant with ascarosides or providing ascarosides in the growth medium of a plant to prevent or inhibit pathogen growth inside the growing plant.

The compositions and methods of the invention control pathogens, particularly food borne human pathogens. The pathogens include human pathogens such as viruses, bacteria, fungi, parasites, and/or oomycetes. Representative food pathogens include germs that cause illnesses such as Salmonella, Clostridium perfringens, Campylobacter, Staphylococcus aureus (Staph), Escherichia coli, Listeria, and the like.

In certain embodiments, the seeds that are treated with the ascarosides are seeds of a plant that can produce edible sprouts and/or leafy greens. Seeds that can produce marketable edible sprouts include, but are not limited to, seeds of edible legume crops, seeds of edible cereal crops, seeds of edible oilseed crops, seeds of edible cabbage family crops, seeds of edible grasses, seeds of edible parsley crops, seeds of edible onion family crops, seeds of edible vegetables, and seeds of edible herbs. Leafy greens include, but are not limited to, kale, microgreens, collard greens, spinach, cabbage, beet greens, mustard greens, watercress, lettuce, chard, arugula, endive, bok choy, turnip greens, broccoli, cilantro, and the like. In certain embodiments, the seeds of the plant that are treated one or more ascarosides are seeds of a crop plant.

In one aspect, the present invention provides a method of inhibiting human pathogenic bacterial growth in or on a plant, the method comprising treating seeds of the plant with one or more ascarosides treating seeds of the plant with one or more ascarosides. In certain embodiments, plants grown from such seeds are characterized in that they are more resistant to the proliferation of human pathogenic bacteria upon or within their tissues relative to plants grown under the same conditions from untreated seeds.

In another aspect, the present invention provides a method of inhibiting human pathogenic bacterial growth in or on a plant, the method comprising sprouting seeds of the plant in a growth medium fortified with one or more ascarosides. In certain embodiments, plants grown in such media are characterized in that they are more resistant to the proliferation of human pathogenic bacteria upon or within their tissues relative to plants grown under the same conditions with unfortified media.

In another aspect, the present invention provides a method of inhibiting human pathogenic bacterial growth in or on a plant, the method comprising treating seeds of the plant with one or more ascarosides and sprouting those seeds in a growth medium that is fortified with one or more ascarosides. In certain embodiments, plants grown in such conditions are characterized in that they are more resistant to the proliferation of human pathogenic bacteria upon or within their tissues relative to plants grown from untreated seeds using unfortified media.

Ascarosides are secondary metabolites produced by nematodes. A large number of structurally diverse ascaroside structures have been identified in nature and the molecules are believed to function as an evolutionarily conserved chemical language used by nematodes to control many aspects of their development. Ascarosides are also perceived by other organisms and have been demonstrated to have a range of effects on numerous organisms including: bacteria, fungi, plants, and mammals including humans. In the present disclosure, the application of ascarosides to the seeds of plants is shown to activate and prime plant defenses, conferring protection against colonization and/or proliferation by non-plant pathogens, including human pathogenic bacteria. Such protection includes preventing pathogen proliferation within tissues of the growing plant. The methods of the invention to stimulate the plant's innate defense systems are a unique tool to combat pathogens that have infiltrated seeds or internal plant tissues and which are difficult to eradicate using current methods. Once stimulated by the compositions of the invention, the defense responses in seed-treated plants protect against pathogens for several weeks post-germination, which is longer than needed for commercial sprout production which is generally completed in 3-7 days.

Ascarosides are derivatives of the sugar ascarylose—a di-deoxy sugar lacking hydroxyl groups at its 3- and 6-positions. Ascarosides have the general structure shown in Formula I:

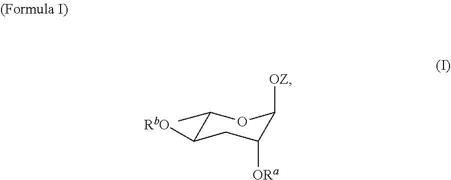

wherein:
Z is an optionally substituted C₂₄₀ aliphatic group, and
each of R^{a} and R^{b} is independently —H, or an optionally substituted moiety selected from the group consisting of: C₁₋₂₀ aliphatic, C₁₋₂₀ acyl, C₁₋₂₀ heteroaliphatic, aryl, heteroaryl, a hydroxyl protecting group, a phosphorous-linked functional group, a sulfur-linked functional group, a silicon-linked functional group, a C₂₋₂₀ carbonate (e.g., a moiety —C(O)OR^{c}), a C₂₋₂₀ carbamate (e.g., a moiety —C(O)N(R^{c})₂), a C₂₋₂₀ thioester (e.g., a moiety —C(S)R^{c}), a C₂₋₂₀ thiocarbonate (e.g., a moiety —C(S)OR^{c}), a C₂₋₂₀ dithiocarbonate (e.g., a moiety —C(S)SR^{c}), a C₁₋₂₀ thiocarbamate (e.g., a moiety —C(S)N(R^{c})₂), a sugar moiety, a peptide, a polymer chain, or a linkage via a bond or a carbon-containing linker moiety to another ascaroside molecule. Where R^{c} is independently at each occurrence selected from —H, optionally substituted C₁₋₁₂ aliphatic, optionally substituted C₁₋₁₂ heteroaliphatic, optionally substituted aryl, optionally substituted heteroaryl, a polymer chain, or a linkage via a bond or a carbon-containing linker moiety to another ascaroside molecule, and where R^{a} and R^{b} may be taken together to form an optionally substituted ring, optionally containing one or more heteroatoms, and optionally containing one or more sites of unsaturation.

In certain embodiments, Z is:
(i) —CH(CH₃)—R¹, where R¹ is an optionally substituted C₁₋₄₀ aliphatic group;
(ii) —CH(CH₃)—(CH₂)ₙ—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(iii) —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(iv) —CH(CH₃)—(CH₂)ₙ—CH(OH)—CH—CO₂R³², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(v) —CH(CH₃)—(CH₂)ₙ—C(O)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(vi) —(CH₂)ₙ—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(vii) —(CH₂)ₙ—CH═CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(viii) —(CH₂)ₙ—CH(OH)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule; or
(ix) —(CH₂)ₙ—C(O)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule.

In certain embodiments, Z is:
(x) —CH(CH₃)—(CH₂)ₙ—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xi) —CH(CH₃)—(CH₂)ₙ—CH═CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xii) —CH(CH₃)—(CH₂)ₙ—CH(OH)—CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xiii) —CH(CH₃)—(CH₂)ₙ—C(O)—CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xiv) —(CH₂)ₙ—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xv) —(CH₂)ₙ—CH═CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xvi) —(CH₂)ₙ—CH(OH)—CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule; or
(xvii) —(CH₂)ₙ—C(O)—CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule.

In certain embodiments, R^{a} is —H.

In certain embodiments, R^{a} is —H. In certain embodiments R^{b} is —H. In certain embodiments, R^{a} and R^{b} are the same. In certain embodiments R^{a} and R^{b} are both —H.

In certain embodiments, R^{a} and R^{b} are different. In certain embodiments, R^{a} is —H, and R^{b} is other than —H. In certain embodiments, R^{a} is other than —H and R^{b} is —H. In certain embodiments, R^{a} is —H and R^{b} is p-hydroxybenzoate. In certain embodiments, R^{a} is —H and R^{b} is indole-3-carboxylate. In certain embodiments, R^{a} is —H and R^{b} is (E)-2-methyl-2-butenoate. In certain embodiments, R^{a} is —H and R^{b} is picolinate. In certain embodiments, R^{a} is —H and R^{b} is nicotinate. In certain embodiments, R^{a} is —H and R^{b} is (R)-2-hydroxy-2-(4-hydroxyphenyl)ethyl)amino)-4-oxobutanoate. In certain embodiments, R^{a} is —H and R^{b} is 4-((4-hydroxyphenethyl)amino)-4-oxobutanoate.

In certain embodiments R^{a} and R^{b} are both —H, and Z is selected from the formulae defined in (i) to (ix) above. In certain embodiments R^{a} and R^{b} are both —H, and Z conforms to formula (i) above. In certain embodiments R^{a} and R^{b} are both —H, and Z conforms to formula (ii) above. In certain embodiments R^{a} and R^{b} are both —H, and Z conforms to formula (iii) above. In certain embodiments R^{a} and R^{b} are both —H, and Z conforms to formula (iv) above. In certain embodiments R^{a} and R^{b} are both —H, and Z conforms to formula (v) above. In certain embodiments R^{a} and R^{b} are both —H, and Z conforms to formula (vi) above. In certain embodiments R^{a} and R^{b} are both —H, and Z conforms to formula (vii) above. In certain embodiments R^{a} and R^{b} are both —H, and Z conforms to formula (viii) above. In certain embodiments R^{a} and R^{b} are both —H, and Z conforms to formula (ix) above. In certain embodiments R^{a} and R^{b} are both —H, and Z conforms to formula (x) above. In certain embodiments R^{a} and R^{b} are both —H, and Z conforms to formula (xi) above. In certain embodiments R^{a} and R^{b} are both —H, and Z conforms to formula (xii) above. In certain embodiments R^{a} and R^{b} are both —H, and Z conforms to formula (xiii) above. In certain embodiments R^{a} and R^{b} are both —H, and Z conforms to formula (xiv) above. In certain embodiments R^{a} and R^{b} are both —H, and Z conforms to formula (xv) above. In certain embodiments R^{a} and R^{b} are both —H, and Z conforms to formula (xvi) above. In certain embodiments R^{a} and R^{b} are both —H, and Z conforms to formula (xvii) above.

In certain embodiments, R² is —H. In certain embodiments, R² is a metal cation. In certain embodiments, R² is an organic cation (e.g., a nitrogen or phosphorous centered cationic group). In certain embodiments, R² is an optionally substituted C₁₋₂₀ aliphatic group. In certain embodiments, R² is an optionally substituted C₁₋₁₂ aliphatic group. In certain embodiments, R² is an optionally substituted C₁₋₈ aliphatic group. In certain embodiments, R² is an optionally substituted C₁₋₆ aliphatic group. In certain embodiments, R² is selected from methyl, ethyl, n-propyl, i-propyl, n-butyl, sec-butyl, and t-butyl. In certain embodiments, R² is an optionally substituted aromatic group. In certain embodiments, R² is a glycoside. In certain embodiments, R² comprises an amino acid. In certain embodiments, R² comprises a peptide. In certain embodiments, R² comprises a nucleotide.

In certain embodiments, at least one R³ is —H. In certain embodiments, both R³ groups are —H. In certain embodiments, at least one R³ is an optionally substituted C₁₋₂₀ aliphatic group. In certain embodiments, both R³ groups are an optionally substituted C₁₋₂₀ aliphatic group which may be the same or different. In certain embodiments, at least one R³ is an optionally substituted C₁₋₁₂ aliphatic group. In certain embodiments, at least one R³ is an optionally substituted C₁₋₈ aliphatic group. In certain embodiments, at least one R³ is an optionally substituted C₁₋₆ aliphatic group. In certain embodiments, at least one R³ is selected from methyl, ethyl, n-propyl, i-propyl, n-butyl, sec-butyl, and t-butyl. In certain embodiments, at least one R³ is —CH₂CH₂OH. In certain embodiments, at least one R³ is —CH₂CH₂OR². where R² is as defined in the genera and subgenera herein. In certain embodiments, at least one R³ is an optionally substituted aromatic group. In certain embodiments, at least one R³ comprises a glycoside. In certain embodiments, at least one R³ comprises an amino acid. In certain embodiments, at least one R³ at least one R³ comprises a peptide. In certain embodiments, at least one R³ comprises a nucleotide.

In certain embodiments, an ascaroside is selected from the group consisting of:

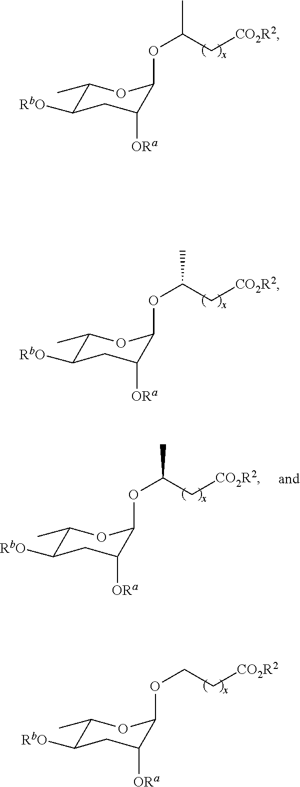

where x is an integer from 1 to 22, and each of R^{a}, R^{b}, and R² is as defined above and in the genera and subgenera herein.

In certain embodiments, an ascaroside is selected from the group consisting of:

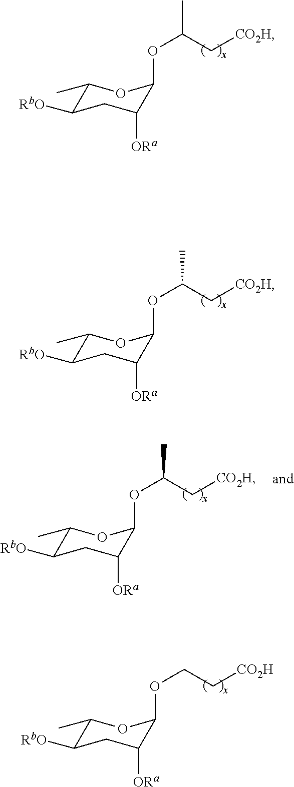

Where each of x, R^{a}, and R^{b}, is as defined above and in the genera and subgenera herein.

In certain embodiments, an ascaroside is selected from the group consisting of:

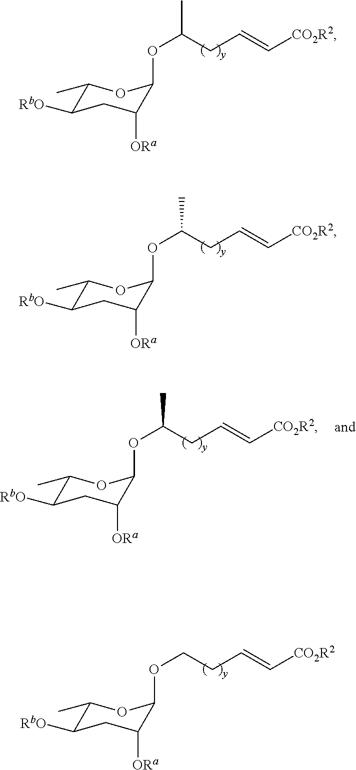

where y is an integer from 1 to 20, and each of R^{a}, R^{b}, and R² is as defined above and in the genera and subgenera herein.

In certain embodiments, an ascaroside is selected from the group consisting of:

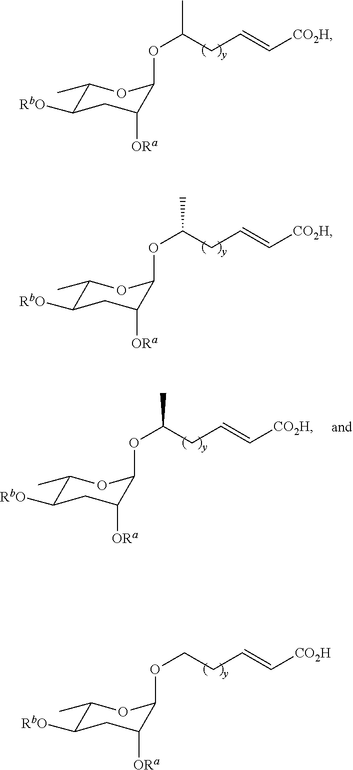

where each of y, R^{a}, and R^{b}, is as defined above and in the genera and subgenera herein.

In certain embodiments, an ascaroside is selected from the group consisting of:

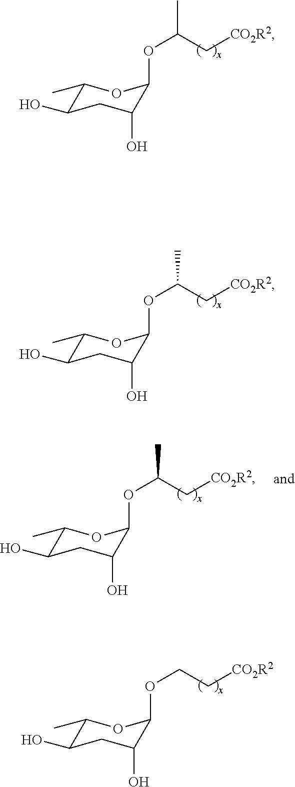

where x is an integer from 1 to 22, and R² is as defined above and in the genera and subgenera herein.

In certain embodiments, an ascaroside is selected from the group consisting of:

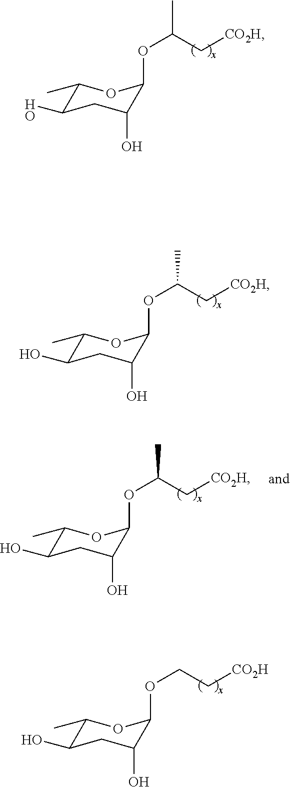

where x is as defined above and in the genera and subgenera herein.

In certain embodiments, an ascaroside is selected from the group consisting of:

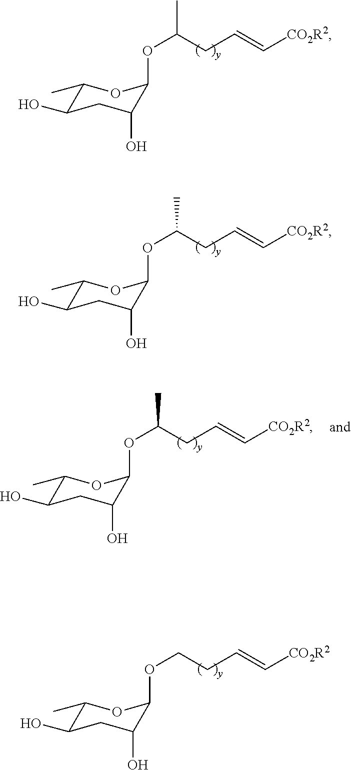

where y is an integer from 1 to 20, and R² is as defined above and in the genera and subgenera herein.

In certain embodiments, an ascaroside is selected from the group consisting of:

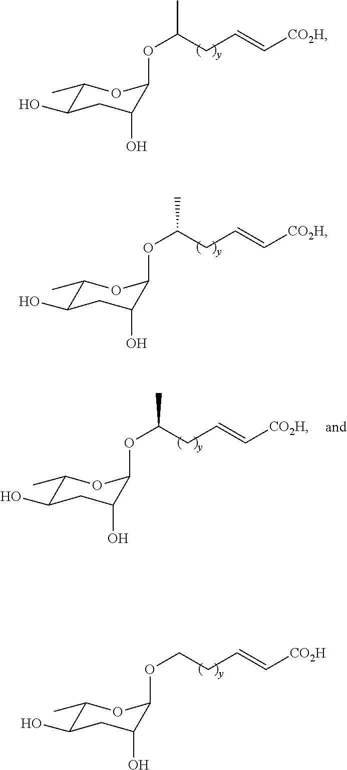

where y is as defined above and in the genera and subgenera herein.

In certain embodiments, an ascaroside is selected from the group consisting of:

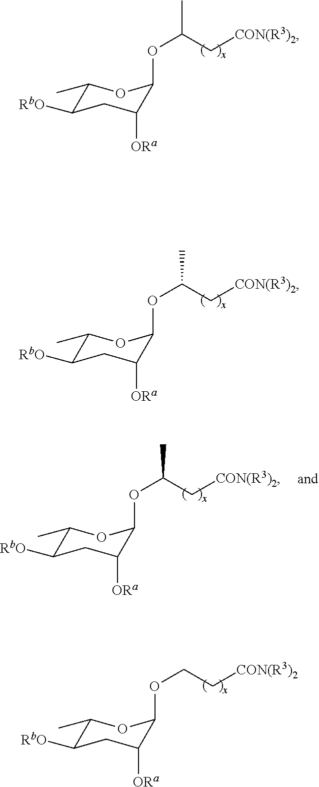

where x is an integer from 1 to 22, and each of R^{a}, R^{b}, and R³ is as defined above and in the genera and subgenera herein.

In certain embodiments, an ascaroside is selected from the group consisting of:

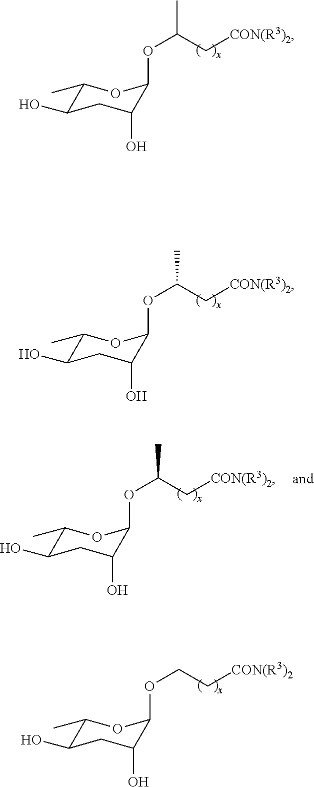

where each of x and R³ is as defined above and in the genera and subgenera herein.

In certain embodiments, an ascaroside is selected from the group consisting of:

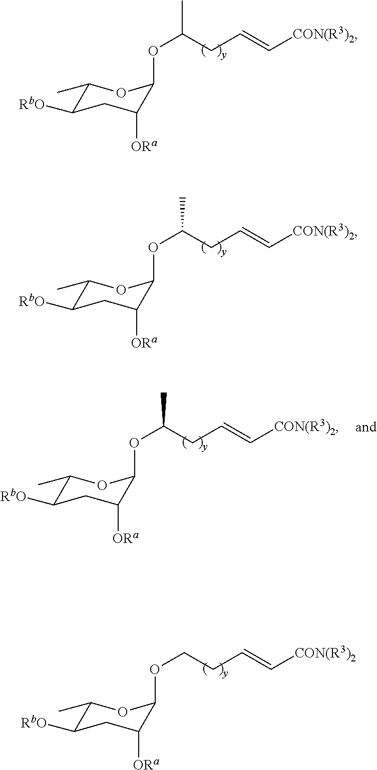

where y is an integer from 1 to 20, and each of R^{a}, R^{b}, and R² is as defined above and in the genera and subgenera herein.

In certain embodiments, an ascaroside is selected from the group consisting of:

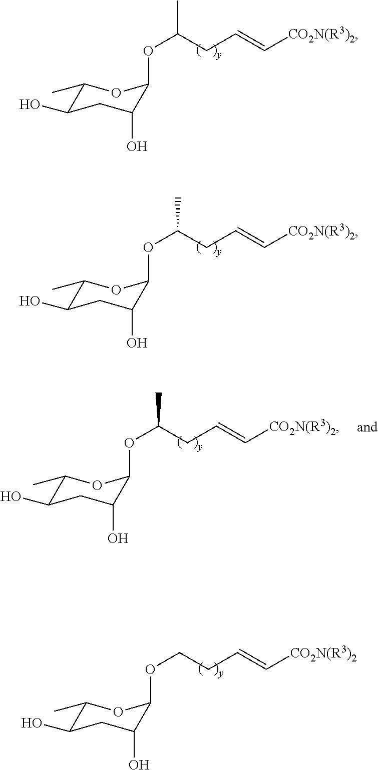

where each of y and R³ is as defined above and in the genera and subgenera herein.

In an embodiment, ascarosides useful in the context of the present disclosure have the general structure (I), where Z is —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide, and can be used for inhibiting human pathogenic bacterial growth in or on a plant.

In an embodiment, ascarosides useful in the context of the present disclosure have the general structure (I) where Z is —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

In an embodiment, ascarosides useful for the present invention have the general structure (I), where Z is —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide, can be used for inhibiting human pathogenic bacterial growth in or on a plant.

In an embodiment, ascarosides useful for the present invention have the general structure (I) where Z is —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

Specific ascarosides that are useful for the present invention include, but are not limited to, ascr #7 and ascr #18.

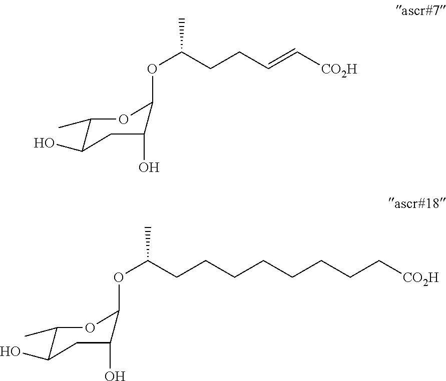 [SMILES: CC1OC(O[C@H](C)CC/C=C/C(=O)O)C(O)CC1O ; CC1OC(O[C@H](C)CCCCCCCCC(=O)O)C(O)CC1O]

In certain embodiments, an ascaroside used in the provided methods is selected from the group consisting of: ascr #9, ascr #12, ascr #14, ascr #1, ascr #10, ascr #16, ascr #18, ascr #20, ascr #22, ascr #24, ascr #26, ascr #28, ascr #30, ascr #32, ascr #34, and ascr #36. In certain embodiments, an ascaroside used in the provided methods is selected from the group consisting of: ascr #10, ascr #16, ascr #18, ascr #20, ascr #22, and ascr #24. In certain embodiments, an ascaroside used in the provided methods is selected from the group consisting of: ascr #9, ascr #14, ascr #10, and ascr #18.

In certain embodiments, an ascaroside used in the provided methods is selected from the group consisting of: ascr #5, oscr #9, oscr #12, oscr #1, oscr #14, oscr #10, oscr #16, oscr #18, oscr #20, oscr #22, oscr #24, oscr #26, oscr #28, oscr #30, oscr #32, oscr #34, and oscr #36. In certain embodiments, an ascaroside used in the provided methods is selected from the group consisting of: oscr #10, oscr #16, oscr #18, oscr #20, and oscr #22. In certain embodiments, an ascaroside used in the provided methods is selected from the group consisting of: bhas #5, oscr #9, oscr #12, oscr #1, oscr #14, oscr #10, oscr #16, oscr #18, oscr #20, oscr #22, oscr #24, oscr #26, oscr #28, oscr #30, oscr #32, oscr #34, and oscr #36. In certain embodiments, an ascaroside used in the provided methods is selected from the group consisting of: oscr #10, oscr #16, oscr #18, oscr #20, and oscr #22.

In certain embodiments, an ascaroside used in the provided methods is selected from the group consisting of: bhas #9, bhas #10, bhas #16, bhas #18, bhas #22, bhas #24, bhas #26, bhas #28, bhas #30, bhas #32, bhas #34, bhas #36, bhas #38, bhas #40, and bhas #42.

In certain embodiments, an ascaroside used in the provided methods is selected from the group consisting of: bhos #10, bhos #16, bhos #18, bhos #22, bhos #24, bhos #26, bhos #28, bhos #30, bhos #32, bhos #34, bhos #36, bhos #38, bhos #40, and bhos #42.

In certain embodiments, an ascarosides used in the provided methods is selected from the group consisting of: ascr #18, oscr #16, oscr #17, oscr #15, bhas #18, bhos #16, glas #18, dhas #18, ibha #18, ibho #16, icas #18, icos #15, icos #16, and any combination of two or more of these.

Ascarosides can be obtained from natural sources (e.g., nematodes) or they may be prepared synthetically. Ascarosides can be prepared synthetically, for example, by converting 1-O-substituted rhamnose to 1-O-substituted ascarylose. An exemplary method of preparing ascarosides includes: providing as a feedstock a 1-O-substituted rhamnose; forming a mono-sulfonate ester at the 3-OH group of the feedstock; and treating the mono-sulfonate ester with a hydride source to form a 1-O-substituted ascarylose. In certain embodiments, forming the mono-sulfonate ester is conducted on a substrate without hydroxyl protecting groups at the 2- or 4-position of the rhamnose feedstock. In certain embodiments, such methods comprise contacting the feedstock with a sulfonating agent (i.e., a sulfonyl halide, sulfonic anhydride or similar reagent) in the presence of a Lewis acid. Specific details regarding the synthesis of 1-0-substituted ascarylose can be found in PCT Application No. PCT/IB2021/056981, filed on Mar. 2, 2022 (published as WO2022/024067), which is incorporated herein by reference.

For the synthesis of ascarosides, it may be desirable and efficient in some embodiments to utilize a rhamnose feedstock having a substituent on the 1-OH position that is identical to the desired substituent of the target ascaroside, or which is a convenient synthetic precursor to the desired substituent. When a 1-O-substituted rhamnose is used as the precursor, the method of making the ascarosides can begin with direct reaction of the 1-O-substituted rhamnose or can comprise a first step of converting rhamnose to the 1-O-substituted rhamnose. Methods of converting OH to O—Z, providing the 1-O-substituted rhamnose are dependent upon the Z substituent, and are generally known in the art.

Prior to use, the ascarosides used in the compositions of the invention may be dissolved in water to form an aqueous solution of the ascaroside. Some ascarosides, however, have limited solubility in water. Therefore, it may be necessary to initially dissolve the ascaroside in a co-solvent that is water miscible and acceptable for use in plant material intended for human consumption. Various water-miscible solvents are known and can be used for this purpose. A preferred co-solvent is ethanol. Another preferred co-solvent is propylene glycol. The water-miscible solvents may be food grade solvents. In an embodiment, a stock solution of the ascaroside dissolved in ethanol or propylene glycol can be prepared. The stock solution is added to a suitable amount of water to prepare the aqueous solution of the ascaroside. As used herein, an “aqueous solution” includes solutions comprising primarily water as the solvent, optionally including one or more co-solvents in an amount up to about 45% by volume, up to about 40% by volume, up to about 35% by volume, up to about 30% by volume, up to about 25% by volume, up to about 20% by volume, up to about 15% by volume, up to about 10% by volume, up to about 5% by volume, up to about 1% by volume, or up to about 0.1% by volume.

In one embodiment, seeds of a plant may be treated with an ascaroside solution (e.g., an aqueous solution of one or more ascarosides) by soaking the seeds in the ascaroside solution. The seeds are soaked in the ascaroside solution for a time sufficient to allow ascarosides to enter into the seed. Typically, the seeds are soaked for a time of about 1 min. to about 12 hours (e.g., about 5 min. to about 6 hours or about 30 min. to about 3 hours, or about 6 hours to about 12 hours), although such times are not intended to be limiting, and greater or lesser time ranges can, in some embodiments, be employed. In certain embodiments, the seeds are soaked in a solution containing one or more ascarosides for about 1 min., about 5 min., about 10 min., about 20 min., about 30 min., about 45 min., about 1 hour, about 2 hours, about 3 hours, about 4 hours, about 5 hours, about 6 hours, about 7 hours, about 8 hours, about 9 hours, about 10 hours, about 11 hours, or about 12 hours.

The change in the weight of the seeds can be used to determine if the seeds have been soaking in the ascaroside solution for a sufficient amount of time to allow the ascarosides to enter the seeds. Prior to soaking the seeds in the ascaroside solution, the seeds are weighed to determine the “dry weight” of the seeds. The seeds are then soaked in the ascaroside solution for a predetermined amount of time (e.g., the times noted above). The seeds are removed from the ascaroside solution and dried to remove all or a portion of the water that is on the surface of the seeds. The dried seeds are weighed to determine the amount of water absorbed into the seed while soaking in the ascaroside solution. A substantial, positive, change in weight of the seeds indicates that ascaroside solution was absorbed by the seeds. A change in weight of greater than 2%, greater than 3%, greater than 4%, greater than 5%, greater than 6%, greater than 8%, greater than 10%, greater than 12%, greater than 15%, or greater than 20% indicates the absorption of a sufficient amount of the ascaroside solution.

The ascaroside solution has an ascaroside concentration ranging from 0.001 mM to about 1.0 mM (e.g., about 0.01 mM to about 0.5 mM, about 0.1 mM to about 0.5 mM, or about 0.5 to about 1 mM). When the ascaroside solution includes more than one ascaroside, the phrase “ascaroside concentration” refers to the total concentration of all ascarosides in the solution. Exemplary ascaroside concentrations in an ascaroside solution that is used to treat seeds include 0.001 mM, 0.005 mM, 0.01 mM, 0.05 mM, 0.1 mM, 0.5 mM, and 1.0 mM.

In one embodiment, seeds of a plant may be treated with an ascaroside seed coating formulation (e.g., a liquid or powder composition containing one or more ascarosides). Seed coating may be achieved by any known means including spraying the composition onto the seeds, or agitating the seeds with the composition. In contrast to soaking, these methods generally use a minimum amount of liquid and the seeds are generally dried after application to provide viable treated or coated seeds that are stable to storage and transport. In certain embodiments, such seed treatments are applied at rates to provide treated seed having from about 0.01 ppm to about 100 ppm ascaroside content by weight. In certain embodiments, such seed contains from about 0.1 to about 10 ppm ascarosides. In certain embodiments, such seed contains from about 0.2 to about 5 ppm ascarosides. In certain embodiments, such seed contains from about 1 to about 10 ppm ascarosides. In certain embodiments, such seed contains from about 2 to about 5 ppm ascarosides. In certain embodiments, such seed contains from about 0.1 to about 1 ppm ascarosides. In certain embodiments, such seed contains from about 0.5 to about 2.5 ppm ascarosides. In certain embodiments, the method includes treating the seed, drying the treated seed, storing the seed, and optionally transporting the seed to a production facility prior to producing sprouts from the treated seed.

In a typical process for the growth of sprouts, seeds are soaked in water to increase the water content in the seed and to bring the seeds out of quiescence. It may therefore be convenient to treat the seeds with the ascaroside composition during this soaking step of sprout production. The seeds are then placed in a container that allows the seeds to be periodically rinsed with water without the seeds being removed from the container. The seeds are generally rinsed two to four times a day, depending on the type of seed. This provides the seeds with enough moisture to sprout and grow. In certain embodiments, ascarosides may be included in one or more of these rinses. Depending on the type of plant associated with the seeds, the sprouts are cultivated after growing to about 5 to 8 centimeters. As previously discussed, this process of producing sprouts for consumption can be conducive for bacterial proliferation.

There are many different kinds of sprouts that are cultivated and consumed. Examples of general classes of edible sprouts include, but are not limited to, legume family sprouts, cereal family sprouts, oilseed family sprouts, cabbage family sprouts, parsley family sprouts, onion family sprouts, vegetable sprouts, and herb sprouts. Examples of legume family sprouts include, but are not limited to, mung bean sprouts, soybean sprouts, alfalfa sprouts, clover sprouts, fenugreek sprouts, lentil sprouts, pea sprouts, and chickpea sprouts. Examples of cereal family sprouts include, but are not limited to, oat sprouts, corn sprouts, rice sprouts, barley sprouts, and rye sprouts. Examples of oilseed family sprouts include, but are not limited to, sesame sprouts, sunflower sprouts, almond sprouts, hazelnut sprouts, hemp sprouts, linseed sprouts, and peanut sprouts. Examples of cabbage family sprouts include, but are not limited to, broccoli sprouts, cabbage sprouts, watercress sprouts, mustard sprouts, mizuna sprouts, radish sprouts, daikon sprouts, arugula sprouts, tatsoi sprouts and turnip sprouts. Examples of parsley family sprouts include, but are not limited to, carrot sprouts, celery sprouts, fennel sprouts, and parsley sprouts. Examples of onion family sprouts include, but are not limited to, onion sprouts, leek sprouts, green onion sprouts. Examples of vegetable and herb sprouts include, but are not limited to, spinach sprouts, lettuce sprouts, milk thistle sprouts, and lemon grass sprouts.

In an embodiment of the present invention, it has been found that soaking the seeds from a plant that is used to form an edible sprout in an ascaroside solution can inhibit the formation of pathogenic bacteria in and on the sprouts during growth and cultivation of the sprouts. While not wishing to be held to any particular theory, it is believed that the protective effects of treatment with ascarosides results from the upregulation of plant defense genes by the presence of the ascarosides.

Biofilm formation represents one of the major defense strategies employed by bacteria against hostile environmental conditions. Biofilm formation by bacteria on the exterior or interior of sprouts can provide a protected environment where pathogenic bacteria can colonize and thrive. While not being bound to any particular theory, it is believed that that ascarosides can affect bacteria's ability to form biofilms. By disrupting biofilm formation, ascarosides can remove one of the bacteria's primary defense mechanisms.

A variety of human pathogenic bacteria can be controlled by the treatment of seed or plants with an ascaroside solution. Examples of bacteria that can be controlled include, but are not limited to, Salmonella enterica, Salmonella serovar Muenchen, Salmonella Saintpaul, Escherichia coli O157:1H 7, Escherichia coli O157:NM, Escherichia coli O121, Escherichia coli O104:H4, and Listeria monocytogenes.

Based on the weight change of the seeds after soaking in the ascaroside solution, it is theorized that the seeds uptake about 0.01 to about 100 ppm of the ascarosides after soaking in the ascaroside solution. The amount of uptake of ascaroside in the seeds is a function of the concentration of ascarosides in the ascaroside solution and the duration of soaking. The amount of ascaroside that is present in the treated seeds can therefore be modified by adjusting these parameters. Typically, seeds are soaked in the ascaroside solution at ambient temperature and pressure. In some embodiments, seeds may be soaked in an ascaroside solution that is at a temperature and/or pressure that is higher than ambient temperature and/or pressure. Treatment of seeds at a temperature and/or pressure that is higher than ambient temperature and/or pressure can kill some or all human pathogenic bacteria that may be present in and/or on the seeds.

While particularly useful for treating seeds that will be used for sprout production, the method of treating seeds with an ascaroside solution can also be used for treating crop plant seeds. Crop plants having seeds that can be treated with an ascaroside solution include, but are not limited to, corn, wheat, rice, soybean, tomato, lettuce, potato, barley, and beans. The method of treating crop plant seeds is the similar to the method of treating seeds for sprout seeds. The crop plant seeds are soaked in an ascaroside solution for a time sufficient to allow ascarosides to enter into the seeds. The time of soaking and composition and concentration of the ascaroside solution are similar, or the same, as discussed above. After treatment with the ascaroside solution the seeds may be planted into the ground in a manner typical for the particular crop plant. Crop plants that are grown from seeds treated with an ascaroside solution can show better resistance to both human pathogenic bacteria as well as bacteria and fungi that can affect the health or growth of the plant.

In an alternate method of production of sprouts, seeds from a plant that is used to produce edible sprouts are treated by soaking the seeds in an ascaroside solution for a time sufficient to bring the seeds out of quiescence (i.e., the seeds have begun germination). In this method the initial soaking in the ascaroside solution accomplishes both the infiltration of ascarosides into the seeds and the initial step of the sprout formation process. After soaking the seeds for a time sufficient to ensure that the seeds are in the germination state, the seeds are placed in an appropriate container for sprout formation. The seeds are then rinsed periodically until the sprouts reach the appropriate length. In a related alternate method of production of sprouts, ascarosides are included in the water used to periodically rinse the sprouts. The ascarosides may be present in each rinse, only one rinse, or in some of the rinses (i.e., where one or more treatments with ascarosides are alternated with one or more treatments without ascarosides).

Treatment of seeds with an ascaroside solution, unexpectedly, was found to increase the rate of germination of the seed and accelerate growth and development of the plant. In another embodiment, plant seeds, used for either sprout production or crop production, are treated with an ascaroside solution to increase the rate of germination and accelerate growth of the resulting plant. Similar to the other embodiments, the plant seeds are soaked in an ascaroside solution for a time sufficient to allow one or more ascarosides to enter into the seeds. After treatment with the ascaroside solution, the seeds are used for sprout production or crop production, as appropriate. The increase in rate of germination of the seed and accelerated growth and development of the resulting plant can vary. In some embodiments, a significant increase in the rate of germination is observed. In some embodiments, a significant increase in the percentage of germinated seeds (as opposed to ungerminated seeds) within a given sample that germinate is observed. In some embodiments, a significant increase in the rate of growth and/or development of the plant is observed.

In another embodiment, it has been found that an ascaroside solution is effective at inhibiting human pathogenic bacteria growth and/or bacteria and fungi that can affect the health or growth of the plant when the ascaroside solution is sprayed onto the plant. The ascaroside solution used to treat the plants, once the plant has begun growing, may have a concentration that is substantially lower than the concentration of the ascaroside solution that is used to treat seeds. For a typical crop plant or sprouts, the ascaroside solution used for spray treatment of these plants ranges from about 1 ppb up to about 50 ppm. For example, the concentration of ascaroside used for spray treatment of plants may be 1 ppb, 10 ppb, 50 ppb, 100 ppb, 250 ppb, 500 ppb, 1 ppm, 2 ppm, 3 ppm, 5 ppm, 10 ppm, 15 ppm, 20 ppm, 25 ppm, 30 ppm, 40 ppm, or 50 ppm.

The treatment of seeds with an ascaroside solution surprisingly provides protection against bacterial and fungal contamination for at least six weeks. This is particularly useful for crop plants that will require a month or more to yield a crop. This property of the ascaroside solution can be a highly effective preventative for crops. The application of an ascaroside solution to a large quantity of seeds is much easier to perform then spraying entire fields of crops. Treatment of seeds using an ascaroside solution can, in some embodiments, provide enhanced protection against bacterial and fungal contamination for at least one week, for at least two weeks, for at least three weeks, for at least four weeks, for at least five weeks, or for at least six weeks.

Among the leafy greens vegetables, baby leaf vegetables are increasingly popular. While the definition varies, these crops are best defined as vegetables harvested after the development of true leaves, but before the 8 true-leaf stage. A diverse array of leafy green salad crops are grown for baby-leaf salad mixes in the U.S., including lettuce, spinach, mustard greens, pak choi (also known as bok choy), kale, arugula, and beet greens. Baby greens are generally harvested by hand by cutting or pinching the outer, most mature leaves. The injury caused on leaves during harvest becomes an easy entry point for pathogens to contaminate internal plant tissues, where they can remain protected even from post-harvest biocidal washing. Seeds of plants used to produce leafy greens can be treated with an ascaroside solution to protect the plants from pathogen contamination after harvesting. The protection afforded by treatment of the seed of a plant can minimize opportunistic pathogen contamination during harvesting of the leaves of that plant. Such protection may also be imparted to plants after the plants have grown by spraying an ascaroside solution onto the leaves (which can optionally be done in addition to treating the seed with an ascaroside solution, or can be conducted on plants grown from non-treated seeds).

Microgreens are the seedlings of edible plants harvested 7-14 days after planting when the first true leaves start to emerge and thus share characteristics of both sprouts and green leafy vegetables, including the potential to carry foodborne pathogens. Similar to leafy greens, microgreens are also harvested by hand, by cutting just above the soil line and equally prone to pathogen contamination. In an embodiment, seeds of plants used to produce microgreens can be treated with an ascaroside solution to protect the plants from pathogen contamination after harvesting. Such protection can minimize opportunistic pathogen contamination during harvesting of the microgreens. Such protection may also be imparted to plants used for microgreen production after the plants have grown by spraying an ascaroside solution onto the leaves. Again, such application to the leaf can optionally be done in addition to treating the seed with an ascaroside solution, or can be conducted on plants grown from non-treated seeds).

The seed treatments and leaf treatments as provided herein have been shown to reduce or prevent pathogen contamination throughout the early life cycle of the plant. Furthermore, the plant itself has been shown to be protected from pathogen contamination in the event of injury from harvesting and insects. As discussed above, the treatment of seeds and plants with ascarosides appears to upregulate the plant defense genes, allowing the plant to defend itself from pathogen contamination. This type of extended protection helps ensure that the edible products of the plant are protected, whether in sprout form or leaf form.

Additionally, the seed treatments and leaf treatments as provided herein have been shown to reduce or prevent pathogen contamination without the need for additional types of treatment. While the seed treatment and leaf treatment as provided herein can be the only pathogen-reducing treatment conducted, in other embodiments, these treatments can be used in tandem with one or more known methods for reducing or eliminating pathogens including, but not limited to, treatment with acidic electrolyzed water, ozonated water, chlorine dioxide, trisodium phosphate, calcium hypochlorite (bleaching powder), and sodium hypochlorite (bleach).

### EXAMPLES

The following examples embody certain methods of the present invention and are not intended to be limiting.

Treatment of Alfalfa Seeds with Ascr #18 for Preventing Salmonella enterica Contamination

In this study, the effectiveness of ascr #18 seed treatment in preventing proliferation of Salmonella enterica is evaluated. The experiment is performed by comparing the level of contamination resulting from artificial inoculation of ascaroside treated and untreated sproutable seeds with a strain of Salmonella enterica known to be a cause of sprout-related foodborne illness in humans.

Salmonella enterica serotype Stanley, that was previously isolated from an alfalfa sprout-related outbreak of human gastrointestinal infection. Seeds are inoculated, evaluated and propagated as described in Appl. Environ. Microbiol. 2017; 83 (7): e03170-16 doi: 10.1128/AEM.03170-16, and J. Food Prot. (2020) 83 (7): 1218-1226 (doi: 10.4315/JFP-20-021) the entirety of each of which is incorporated herein by reference. Alfalfa seeds (Medicago sativa) are first sanitized to inactivate the background microflora and then soaked in solutions containing various concentrations of ascr #18 (e.g., 0.1 to 1000 uM) or in a mock solution lacking ascr #18. The seeds are then mixed with sterilized sandy soil inoculated with 10⁴ CFU of freeze-dried, nalidixic acid- (NA-) resistant Salmonella cells as described in J. Food Prot. (2020) 83 (7): 1218-1226. Inoculated alfalfa seeds and un-inoculated control seeds (1 g each) were then soaked in solutions with 0.0 mM (mock), 0.001 mM, or 1.0 mM ascr #18, and incubated at room temperature for 20 minutes. An ascr #18 stock solution was prepared by dissolving acr #18 in ethanol to provide a concentrated stock solution that was diluted into distilled water at appropriate ratios to provide the concentrations for seed soaking experiments. To account for any effects caused by the ethanol, the two mock treatments contained amounts of ethanol equivalent to those in the two ascr #18 treatments. Seeds treated with sterile distilled water were included as additional controls. Analysis of the soaked seeds indicated that alfalfa seeds gained ˜8-10% of their dry weight during a 20 min treatment, which translates to the uptake of approximately 0.03 and 30 ppm of ascr #18 in the seeds treated with 0.001 mM and 1.0 mM solutions, respectively.

Salmonella-inoculated alfalfa seeds from the different treatments were plated and allowed to grow for 7 days. Samples were taken every other day for microbiological analyses. For each sample, five seedlings were harvested and ground, and 10-fold serial dilutions from the processed samples were spotted onto Bismuth Sulfite Agar (BSA) or Tryptic Soy Agar media amended with NA (NA-TSA) to quantify the population of Salmonella. In cases where the numbers of Salmonella cells were below the detection limit, enrichment analysis was performed. Two independent trials were conducted and analyzed using Fisher's least significant difference tests (95% confidence interval) to determine the differences in Salmonella populations (log CFU/g of sprout tissues) recovered from the sprouts. The inoculated seeds, and uninoculated control seeds are germinated, grown, sampled and analyzed for presence of Salmonella as described in J. Food Prot. (2020) 83 (7): 1218-1226.

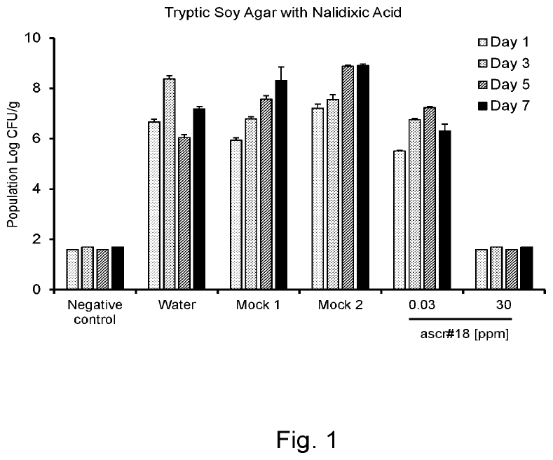

The results are presented in FIG. 1. Two mock solutions were used as controls. Mock 1 is an aqueous solution of ethanol that corresponds to the ethanol concentration present in the 0.001 mM ascr #18 solution. Mock 2 is an aqueous solution of ethanol that corresponds to the ethanol concentration present in the 1.0 mM ascr #18 solution. Salmonella counts on sprouts from alfalfa seeds treated with the 1.0 mM solution (correlating to 30 ppm ascr #18 uptake) were 5.91-7.21 log CFU/g lower than on sprouts of seeds treated with corresponding solvent control (Mock 2) or just water, when plated on NA-TSA. Similar to NA-TSA plates, Salmonella counts from the treatment of seeds with the 1.0 mM ascr #18 solution were found to be 5.61-7.27 log CFU/g lower than control on BSA plates (results not shown). On both media, Salmonella counts for samples treated with the 1.0 mM ascr #18 solution were below the detection limit, even after enrichment, meaning no Salmonella cells were detectable. Seed treatments using the 0.001 mM solution of ascr #18 were less effective than the 1.0 mM ascr #18 solution, but still showed a modest reduction in Salmonella levels ranging from 0.11-2.34 log CFU/g on NA-TSA and 0.03-2.01 log CFU/g on BSA when compared to the corresponding solvent control (Mock 1). In an independent experiment, treatment with an ascr #18 solution provided a similar level of contamination suppression on alfalfa sprouts inoculated with another outbreak-associated strain, Salmonella enterica serotype Cubana (results not shown).

Treatment of seeds with ascr #18 is found to be effective at preventing Salmonella contamination in cultivated sprouts relative to untreated controls.

The ascaroside used to treated seeds was also found to have a short half-life in plants after application and that their persistent effects are the result of activation of defense priming and are not dependent on the continued presence of the active ingredient. These results are encouraging since the ascr #18 content of the produced sprout is 1000-fold less than the seed treatment level. It is anticipated that the residual ascr #18 levels in a commercial setting will be even lower since the minimum effective application rate required for commercial sprout production is likely to be lower than the rate used for these studies.

Unlike other products that activate defense responses in plants, treatment with ascarosides does not negatively impact plant vigor. On the contrary, ascarosides have been found to enhance germination and accelerate the growth and development of some crop plants.

### Ascr #18 Activates Defense Gene Expression in Alfalfa

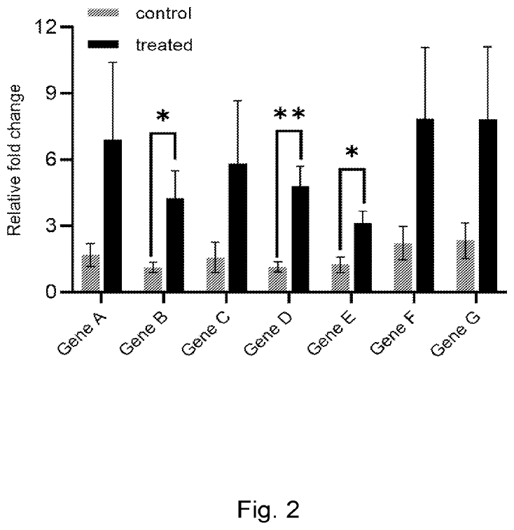

To assess whether the protective effects of ascr #18-based seed treatments result from upregulation of plant defense genes, alfalfa seeds were treated with ascr #18 as above and grown for 7 days. The sprout tissue was analyzed by qRT-PCR for expression of plant defense marker genes including cyclin-dependent protein kinase inhibitor (gene A), RPM1 (gene B), NAC-domain containing protein 72 (gene C), salicylic acid carboxylic methyltransferase 3 (gene D), RPV1-like (gene E), PR-1 like (gene F), and zinc-finger transcription factor (gene G). The results of this study are presented in FIG. 2. Ascr #18 treatment enhanced expression of all seven genes, with three at statistically significant levels. These results indicate a long-term activation of defense responses in alfalfa by ascr #18-based treatments. The expression of these genes was also upregulated in 7-day old alfalfa plants that were sprayed with 1 μM ascr #18 24 h prior to harvesting for RNA analysis (results not shown).

Determination of Residual Ascr #18 on Sprouts Derived from Treated Seeds

A preliminary study was performed to assess residual levels of ascr #18 present in 7-day old sprouts grown from seeds treated with a 1.0 mM solution of ascr #18 as described above. The harvested sprouts were freeze-dried, ground, and extracted with 80:20 methanol:water before being analyzed by liquid chromatography coupled to mass spectrometry (LC-MS). Only trace amounts of ascr #18 (˜30 ppb) could be detected in these sprouts. This is consistent with our previous studies that showed ascarosides have a short half-life in plants after application and that their persistent effects are the result of activation of defense priming and are not dependent on the continued presence of the active ingredient. These results are encouraging since the ascr #18 content of the produced sprout is 1000 fold less than the seed treatment level. It is anticipated that the residual ascr #18 levels in a commercial setting will be even lower than those found in this experiment since the minimum effective application rate required for commercial sprout production is likely to be lower than the rate used for these studies.

### Ascaroside Ascr #18 Promotes Germination and Seedling Vigor

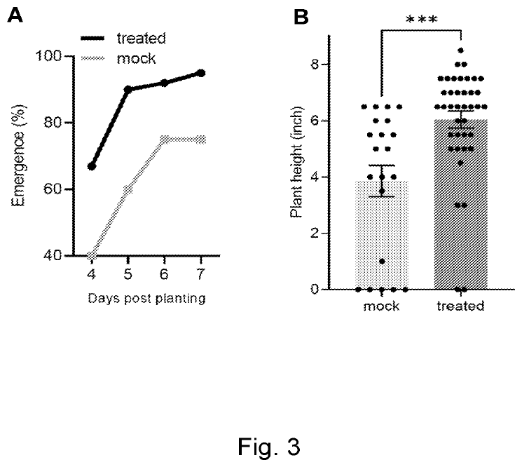

Unlike other products that activate defense responses in plants, treatment with ascr #18 does not negatively impact plant vigor. On the contrary, ascr #18 has been found to enhance germination and accelerate the growth and development of some crop plants. Rice seeds were treated with mock or 1 μM ascr #18 solution by soaking. Emergence was measured at 4, 5, 6, and 7 days post-planting (FIG. 3A). Plant height was measured 10 days post-planting (FIG. 3B). (Data are average±SEM (n≥21). ***P≤0.0004; two-tailed t-test). As shown in FIG. 3, treatment of rice seeds with ppb levels of ascr #18 provided enhanced germination and plant growth. Similar results were obtained from tomato and Arabidopsis (results not shown). Preliminary analysis suggests that ascr #18 treatment has no negative impact on alfalfa germination, growth, and vigor (results not shown).

### Protective Effects of Ascr #18 on Seed-Treated Plants Last at Least Six Weeks Post-Emergence

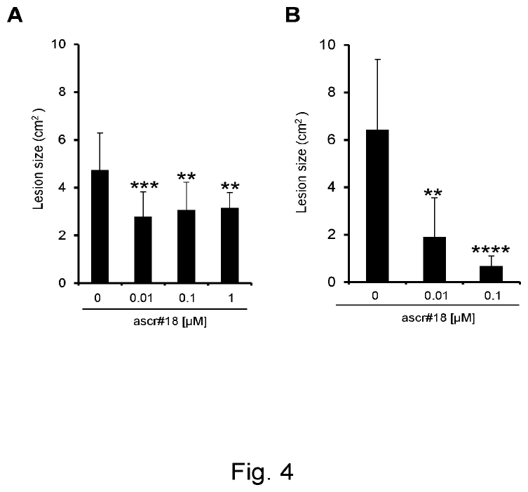

To determine if the defense responses elicited by ascr #18-based seed treatments persist, the impact on resistance against the oomycete pathogen Phytophthora infestans in 6-week-old tomato plants grown from seeds treated with ascr #18 before planting was measured. The results of this test are presented in FIG. 4. FIG. 4A shows the effect of ascr #18 treatment of tomato plant (cv. Sweet hybrid 100) seeds on lesion size caused by the oomycete pathogen. The seeds were treated with 0, 0.01, 0.1 and 1 μM ascr #18. FIG. 4B shows the effect on lesion size on plants produced from tomato plant (cv. M82) seeds that were treated with 0, 0.1, and 0.01 μM ascr #18. Six-week-old plants from both studies were inoculated with P. infestans. Lesion size was measured at 5 dpi. **p<0.005; ***p<0.0005; ****p<0.00005; two-tailed t-test. Enhanced protection, as indicated by the reduction in sporangia lesion size, was similar to that achieved in 6-week-old plants sprayed/drenched with ascr #18 24 h before infection. In a similar study, treatment of soybean seeds with an ascr #18 solution provided protection against Pseudomonas syringae, Phytophthora sojae, and Soybean Mosaic Virus in soybean plants for at least 3-4 weeks (results not shown).

It is contemplated that compositions, systems, devices, methods, and processes of the present application encompass variations and adaptations developed using information from the embodiments described in the present disclosure. Adaptation or modification of the methods and processes described in this specification may be performed by those of ordinary skill in the relevant art.

It will be appreciated that use of headers in the present disclosure are provided for the convenience of the reader. The presence and/or placement of a header is not intended to limit the scope of the subject matter described herein. Unless otherwise specified, embodiments located in one section of the application apply throughout the application to other embodiments, both singly and in combination.

Throughout the description, where compositions, compounds, or products are described as having, including, or comprising specific components, or where processes and methods are described as having, including, or comprising specific steps, it is contemplated that, additionally, there are articles, devices, and systems of the present application that consist essentially of, or consist of, the recited components, and that there are processes and methods according to the present application that consist essentially of, or consist of, the recited processing steps.

It should be understood that the order of steps or order for performing certain action is immaterial so long as the described method remains operable. Moreover, two or more steps or actions may be conducted simultaneously.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A method of inhibiting the growth of human enteric pathogens in a plant, comprising treating seeds of the plant with a composition comprising one or more ascarosides.

2. The method of claim 1, wherein the one or more ascarosides have the structure (I) 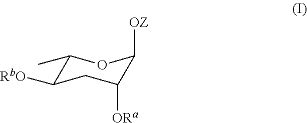
where:
Z is an optionally substituted C₃₋₄₀ aliphatic group, and
each of R^{a} and R^{b} is independently —H, or an optionally substituted moiety selected from the group consisting of: C₁₋₂₀ aliphatic, C₁₋₂₀ acyl, C₁₋₂₀ heteroaliphatic, aryl, heteroaryl, a hydroxyl protecting group, a phosphorous-linked functional group, a sulfur-linked functional group, a silicon-linked functional group, a C₂₋₂₀ carbonate (e.g., a moiety —C(O)OR^{c}), a C₂₋₂₀ carbamate (e.g., a moiety —C(O)N(R^{c})₂), a C₂₋₂₀ thioester (e.g., a moiety —C(S)R^{c}), a C₂₋₂₀ thiocarbonate (e.g., a moiety —C(S)OR^{c}), a C₂₋₂₀ dithiocarbonate (e.g., a moiety —C(S)SR^{c}), a C₁₋₂₀ thiocarbamate (e.g., a moiety —C(S)N(R^{c})₂), a sugar moiety, a peptide, a polymer chain, or a linkage via a bond or a carbon-containing linker moiety to another ascaroside molecule; where R^{c} is independently at each occurrence selected from —H, optionally substituted C₁₋₁₂ aliphatic, optionally substituted C₁₋₁₂ heteroaliphatic, optionally substituted aryl, optionally substituted heteroaryl, a polymer chain, or a linkage via a bond or a carbon-containing linker moiety to another ascaroside molecule, and where R^{a} and R^{b} may be taken together to form an optionally substituted ring, optionally containing one or more heteroatoms, and optionally containing one or more sites of unsaturation.

3. The method of claim 2, wherein Z is selected from the group consisting of:
i. —CH(CH₃)—R¹, where R¹ is an optionally substituted C₁₋₄₀ aliphatic group;
ii. —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide;
iii. —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide;
iv. —CH(CH₃)—(CH₂)ₙ—CH(OH)—CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide;
v. —CH(CH₃)—(CH₂)ₙ—C(O)—CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide;
vi. —(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide;
vii. —(CH₂)ₙ—CH═CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide;
viii. —(CH₂)ₙ—CH(OH)—CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide; and
ix. f-(CH₂)ₙ—C(O)—CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

4. The method of claim 2, wherein R^{a} and R^{b} are each —H.

5. The method of any one of claims 2 to 4, wherein Z is —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

6. The method of claim 1, wherein at least one of the one or more ascarosides is ascr #18.

7. The method of any one of claims 2 to 4, wherein Z is —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

8. The method of claim 1, wherein at least one of the one or more ascarosides is ascr #7.

9. The method of any one of the preceding claims, wherein treating seeds of the plant comprises soaking the seeds in the aqueous solution comprising one or more ascarosides.

10. The method of claim 9, wherein the seeds are soaked in the aqueous solution for a time of about 1 min to about 6 hours.

11. The method of claim 9, wherein the concentration of the one or more ascarosides in the aqueous solution is between about 0.001 mM and about 1.0 mM.

12. The method of any one of the preceding claims, wherein the seeds of the plant are seeds of a plant that can be used to produce edible sprouts.

13. The method of claim 12, wherein the seeds of the plant are seeds that can produce edible legume family sprouts.

14. The method of claim 12, wherein the seeds of the plant are seeds that can produce edible cereal family sprouts.

15. The method of claim 12, wherein the seeds of the plant are seeds that can produce edible oilseed family sprouts.

16. The method of claim 12, wherein the seeds of the plant are seeds that can produce edible cabbage family sprouts.

17. The method of claim 12, wherein the seeds of the plant are seeds that can produce edible parsley family sprouts.

18. The method of claim 12, wherein the seeds of the plant are seeds that can produce edible onion family sprouts.

19. The method of claim 12, wherein the seeds of the plant are seeds that can produce edible vegetable or herb sprouts.

20. The method of claim 12, wherein the seeds of the plant are seeds that can produce crop plants.

21. The method of claim 9, wherein the seeds are soaked in the aqueous solution for a period of time sufficient for the seeds to gain about 2% to about 20% of their dry weight.

22. The method of claim 9, wherein the seeds are soaked in the aqueous solution for a period of time sufficient to bring the seeds out of quiescence.

23. A method of inhibiting the growth of human enteric pathogens in or on a plant comprising spraying the plant with a composition comprising one or more ascarosides.

24. The method of claim 23, wherein the one or more ascarosides have the structure (I) 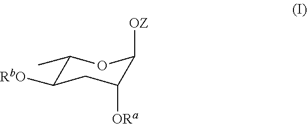
where:
Z is an optionally substituted C₃₋₄₀ aliphatic group, and
each of R^{a} and R^{b} is independently —H, or an optionally substituted moiety selected from the group consisting of: C₁₋₂₀ aliphatic, C₁₋₂₀ acyl, C₁₋₂₀ heteroaliphatic, aryl, heteroaryl, a hydroxyl protecting group, a phosphorous-linked functional group, a sulfur-linked functional group, a silicon-linked functional group, a C₂₋₂₀ carbonate (e.g., a moiety —C(O)OR^{c}), a C₂₋₂₀ carbamate (e.g., a moiety —C(O)N(R^{c})₂), a C₂₋₂₀ thioester (e.g., a moiety —C(S)R^{c}), a C₂₋₂₀ thiocarbonate (e.g., a moiety —C(S)OR^{c}), a C₂₋₂₀ dithiocarbonate (e.g., a moiety —C(S)SR^{c}), a C₁₋₂₀ thiocarbamate (e.g., a moiety —C(S)N(R^{c})₂), a sugar moiety, a peptide, a polymer chain, or a linkage via a bond or a carbon-containing linker moiety to another ascaroside molecule; where R^{c} is independently at each occurrence selected from —H, optionally substituted C₁₋₁₂ aliphatic, optionally substituted C₁₋₁₂ heteroaliphatic, optionally substituted aryl, optionally substituted heteroaryl, a polymer chain, or a linkage via a bond or a carbon-containing linker moiety to another ascaroside molecule, and where R^{a} and R^{b} may be taken together to form an optionally substituted ring, optionally containing one or more heteroatoms, and optionally containing one or more sites of unsaturation.

25. The method of claim 24, wherein R^{a} and R^{b} are both —H.

26. The method of claim 24 or 25, wherein Z is selected from the group consisting of:
i. —CH(CH₃)—R¹, where R¹ is an optionally substituted C₁₋₄₀ aliphatic group;
ii. —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide;
iii. —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide;
iv. —CH(CH₃)—(CH₂)ₙ—CH(OH)—CH—CO₂R³², where n is an integer from 1 to 40, and R² is —H, a metal cation, an optionally substituted C₁₋₂₀ aliphatic group, an optionally substituted aromatic group, a glycoside, an amino acid, a peptide, or a nucleotide;
v. —CH(CH₃)—(CH₂)ₙ—C(O)—CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide;
vi. —(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide;
vii. —(CH₂)ₙ—CH═CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide;
viii. —(CH₂)ₙ—CH(OH)—CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide; and
ix. —(CH₂)ₙ—C(O)—CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

27. The method of claim 24 or 25, wherein Z is —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

28. The method of claim 23, wherein at least one of the one or more ascarosides is ascr #18.

29. The method of claim 24 or 25, wherein Z is —CH(CH₃)—(CH₂)ₙ—CH═CH—CO₂R³, where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

30. The method of claim 23, wherein at least one of the one or more ascarosides is ascr #7.

31. The method of claim 23, wherein a concentration of the one or more ascarosides in the composition is about 1 ppb to about 50 ppm.

32. The method of claim 23, wherein the plant is an edible sprout plant.

33. The method of claim 32, wherein the edible sprout plant is an edible legume family sprout plant.

34. The method of claim 32, wherein the edible sprout plant is an edible cereal family sprout plant.

35. The method of claim 32, wherein the edible sprout plant is an edible oilseed family sprout plant.

36. The method of claim 32, wherein the edible sprout plant is an edible cabbage family sprout plant.

37. The method of claim 32, wherein the edible sprout plant is an edible parsley family sprout plant.

38. The method of claim 32, wherein the edible sprout plant is an edible onion family sprout plant.

39. The method of claim 32, wherein the edible sprout plant is an edible vegetable or herb sprout plant.

40. The method of claim 23, wherein the plant is a crop plant.

41. A method of producing edible sprouts comprising:
treating seeds from a plant that can be used to produce edible sprouts by soaking the seeds in an aqueous solution comprising one or more ascarosides;
placing the treated seeds in a container suitable for allowing sprouts to form from the treated seeds; and
harvesting the sprouts after the sprouts reach a suitable size.
